(19) United States
(12) Patent Application Publication
(10) Pub. No.: US 2026/0185112 A1
(43) Pub. Date: Jul. 2, 2026

(54) METHOD FOR PRODUCING PLANTS WITH ENHANCED CAROTENOID CONTENT USING CRISPR/CAS9-MEDIATED GENE EDITING

(71) Applicant: UNIVERSITY-INDUSTRY COOPERATION GROUP OF KYUNG HEE UNIVERSITY, Yongin-si (KR)

(72) Inventors: Sun-Hwa HA, Suwon-si (KR); Heebak CHOI, Suwon-si (KR)

(73) Assignee: UNIVERSITY-INDUSTRY COOPERATION GROUP OF KYUNG HEE UNIVERSITY, Yongin-si (KR)

(21) Appl. No.: 19/436,675

(22) Filed: Dec. 30, 2025

(30) Foreign Application Priority Data
Dec. 31, 2024 (KR) 10-2024-0202015

(51) Int. Cl. C12N 15/82; C07K 14/415; C12N 9/22; C12N 15/11

(52) U.S. Cl. CPC C12N 15/825; C07K 14/415; C12N 9/226; C12N 15/111; C12N 15/8205; C12N 2310/20

(57) ABSTRACT

The present disclosure relates to a method for producing plants with an enhanced carotenoid content using CRISPR/Cas9-mediated gene editing. It was confirmed that transformed rice was produced using the CRISPR/Cas9 system, in which rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes were deleted, and the carotenoid content was enhanced in the produced transformed rice. Therefore, the present disclosure can be used for producing plants with an enhanced carotenoid content.

### CROSS-REFERENCE TO RELATED APPLICATIONS

This application claims the priority of Korean Patent Application No. 10-2024-0202015 filed on Dec. 31, 2024, in the Korean Intellectual Property Office, the disclosure of which is incorporated herein by reference.

### INCORPORATION BY REFERENCE OF SEQUENCE LISTING

The instant application contains a Sequence Listing which has been filed electronically in xml format and is hereby incorporated by reference in its entirety. Said xml file, created on Dec. 30, 2025, is named Q316162_sequence listing as filed.xml and is 23,853 bytes in size.

### BACKGROUND

### Field

The present disclosure relates to a method for producing plants with an enhanced carotenoid content using CRISPR/Cas9-mediated gene editing.

### Description of the Related Art

Carotenoids are C40 terpenoid metabolites that play an important role in plants as photosynthetic pigments, photoprotectors in the xanthophyll cycle, and precursors of apocarotenoid hormones such as abscisic acid and strigolactone (Walter and Strack, 2011; Nisar et al., 2015). In addition, since the carotenoids act as vitamin A precursors and antioxidants (Mayer et al., 2008; Nisar et al., 2015; Giuliano, 2017) and help in physical development and immune health enhancement (Mayer et al., 2008; Sommer and Vyas, 2012), the biological accumulation of carotenoids has become a continuing interest for the improvement of crops used for food and feed purposes (Giuliano (2017). In particular, rice straw left after harvest has been actively used for baling silage, and is made from gramineous plants (rice, barley, corn, sorghum, and other grains) as feed for ruminants such as cattle and sheep. Due to an increase in feed costs along with the surge in global grain prices, livestock farms are preferring baled silage produced in domestic farms. The current size of the domestic market for baled silage is approximately 1.5 trillion won.

Therefore, various individual and combined technologies have been studied to metabolically control the biosynthesis, degradation, isolation, and stability of carotenoids in plants (Zhai et al., 2016; Giuliano, 2017). Previously, in some crops with active metabolisms of nutritionally valuable specific carotenoids such as beta-carotene and zeaxanthin, the increased amount of accumulation thereof was achieved by silencing directly downstream genes of the corresponding carotenoid metabolism pathways, such as beta-carotene hydrolase (BCH) and zeaxanthin epoxidase (ZEP) (Romer et al., 2002; Diretto et al., 2006; Pons et al., 2014). To produce carotenoids in the endosperm of carotenoid-deficient wheat seeds, a bacterial phytoene synthase (CrtB) gene was overexpressed and simultaneously a wheat endogenous BCH gene TaHyd was silenced to increase carotenoids (Zeng et al., 2015). Plants contain 9-cis-apocarotenoid dioxygenases (NCEDs) and carotenoid cleavage dioxygenases (CCDs) that may degrade carotenoids to produce apocarotenoids (Giuliano et al., 2003; Auldridge et al., 2006; Walter and Strack, 2011; Frusciante et al., 2014; Hou et al., 2016). Homologous genes belonging to a CCD group, such as CCD1, CCD2, CCD4, CCD7, and CCD8, exist in various plant origins. Among these, in a negative correlation between the expression of CCD1 or CCD4 and accumulation of carotenoids, it was suggested that CCD played a role in regulating the carotenoid content in various plant species and organs, including Chrysanthemum leaves, potato tubers, orchid flowers, peach fruits, and Arabidopsis seeds (Ohmiya et al., 2006; Campbell et al., 2010; Chiou et al., 2010; Brandi et al., 2011, Gonzalez-Jorge et al., 2013). In corn, one of the major food crops, in the seed endosperm of corn varieties with white endosperm color due to carotenoid accumulation deficiency, the expression of a ZmCCD1 gene is much stronger than that of the seed endosperm of varieties with yellow endosperm color due to accumulation of carotenoids, which has also been confirmed to play a role in regulating the carotenoid content of CCD (Vallabhaneni et al., 2010). For this reason, it was estimated that when genes encoding carotenoid cleavage dioxygenases (CCDs) are silenced, a step of degrading carotenoids into apocarotenoids is blocked, thereby enhancing carotenoid accumulation. In this regard, it has been reported that when rice CCD genes OsCCD4a and OsCCD4b were edited with CRISPR-CAS9 and then knocked out, the functions in regulating the carotenoid content were analyzed, but had no effect on carotenoid accumulation (Yang et al., 2017). However, this report was a result of an attempt to control the expression of OsCCD4a and OsCCD4b in general rice seeds, which are not suitable for properly verifying an enhancing effect of carotenoids, and the paper suggested a need for further research in rice seeds that produce carotenoids such as beta-carotene. Meanwhile, since rice, one of the major food crops, has the characteristic of not accumulating carotenoids at all in the seed endosperm used as a food source compared to other food crops, vitamin A deficiency is serious in low-income countries that use rice as a main food source and thus ‘Golden rice’ has been developed in which beta-carotene, a major precursor of vitamin A, accumulates in the endosperm of rice seeds (Paine et al., 2005; Ye et al., 2000). In order to enhance the beta-carotene content of the ‘Golden rice’, it was attempted to control the accumulation of the carotenoid content of OsCCD1 in the seed endosperm by expressing OsCCD1 in a sense or antisense direction, but neither sense nor antisense expression of OsCCD1 had any effect on carotenoid accumulation in Golden rice seeds. More precisely, the sense expression study was mainly performed and then it was confirmed that OsCCD1 performed the cleavage reaction using apocarotenoids, not carotenoids, as substrates. The antisense expression study did not provide clear evidence to confirm the suppression of OsCCD1 expression, resulting in insufficient results (Ilg et al., 2010). In summary, since there is still a high possibility that the CCD group genes that degrade carotenoids have a function in regulating the carotenoid content, it is considered that research on the development of a method for controlling the carotenoid content using CCD genes and research on the specialized functions of CCD group genes for each plant tissue are highly useful in the development of metabolic engineering methods for enhancing the carotenoid content.

Accordingly, the present inventors produced transformed rice in which rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes were deleted using the CRISPR/Cas9 system, and confirmed that the carotenoid content was enhanced in the produced transformed rice, and then completed the present disclosure.

### PRIOR ARTS

### Patent Documents

Korean Patent Publication No. 10-2021-0123237

### SUMMARY

An object of the present disclosure is to provide transformed rice including a mutation of at least one gene selected from the group consisting of rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes.

Another object of the present disclosure is to provide a guide RNA for editing an OsCCD1, OsCCD4a or OsCCD4b gene, which is at least one selected from the group consisting of guide RNAs of SEQ ID NOs: 4 to 9.

Yet another object of the present disclosure is to provide a recombinant vector for editing an OsCCD1, OsCCD4a or OsCCD4b gene, including: a sequence encoding the guide RNA; and a sequence encoding a Cas9 protein.

Still another object of the present disclosure is to provide a method for producing transformed rice including: editing at least one gene selected from the group consisting of OsCCD1, OsCCD4a, and OsCCD4b by transforming the vector into rice.

Still another object of the present disclosure is to provide a method for producing transformed rice with an enhanced carotenoid content under dark treatment aging-promoting conditions similar to those in which rice in an aging stage after harvest is introduced into baled silage.

In order to achieve the object, an aspect of the present disclosure provides transformed rice including a mutation of at least one gene selected from the group consisting of rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes.

Another aspect of the present disclosure provides a guide RNA for editing an OsCCD1, OsCCD4a or OsCCD4b gene, which is at least one selected from the group consisting of guide RNAs of SEQ ID NOs: 4 to 9.

Yet another aspect of the present disclosure provides a recombinant vector for editing an OsCCD1, OsCCD4a or OsCCD4b gene, including: a sequence encoding the guide RNA; and a sequence encoding a Cas9 protein.

Still another aspect of the present disclosure provides a method for producing transformed rice including: editing at least one gene selected from the group consisting of OsCCD1, OsCCD4a, and OsCCD4b by transforming the vector into rice.

The present disclosure relates to a method for producing plants with an enhanced carotenoid content using CRISPR/Cas9-mediated gene editing. It was confirmed that transformed rice was produced by deleting the rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes using the CRISPR/Cas9 system, and the carotenoid content and antioxidant activity were enhanced in the produced transformed rice. Accordingly, the transformed rice can be usefully used in the related forage industry.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

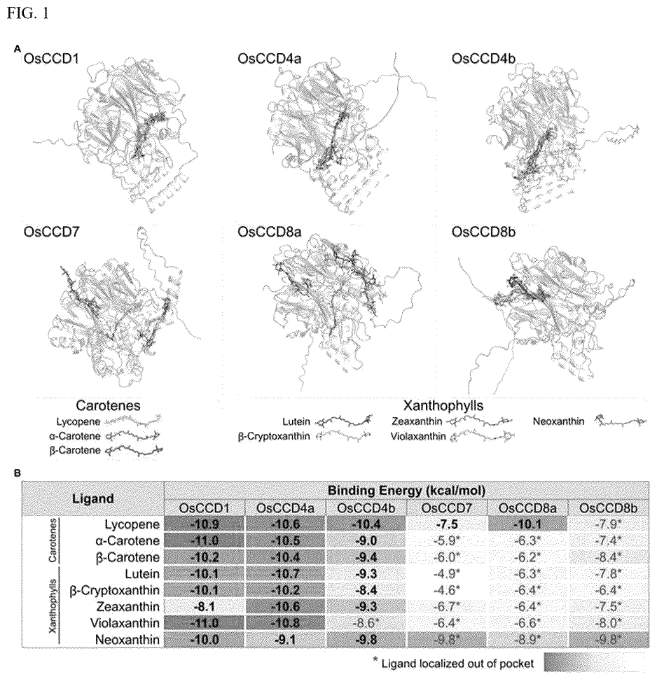

FIG. 1 is a diagram showing results of in silico binding analysis between six CCDs and eight carotenoid ligands (substrates):
A: Molecular docking 3D models of OsCCD1, OsCCD4a, OsCCD4b, OsCCD7, OsCCD8a, and OsCCD8b and predicted binding sites of ligand candidates
B: Binding energy between CCDs and ligand candidates;

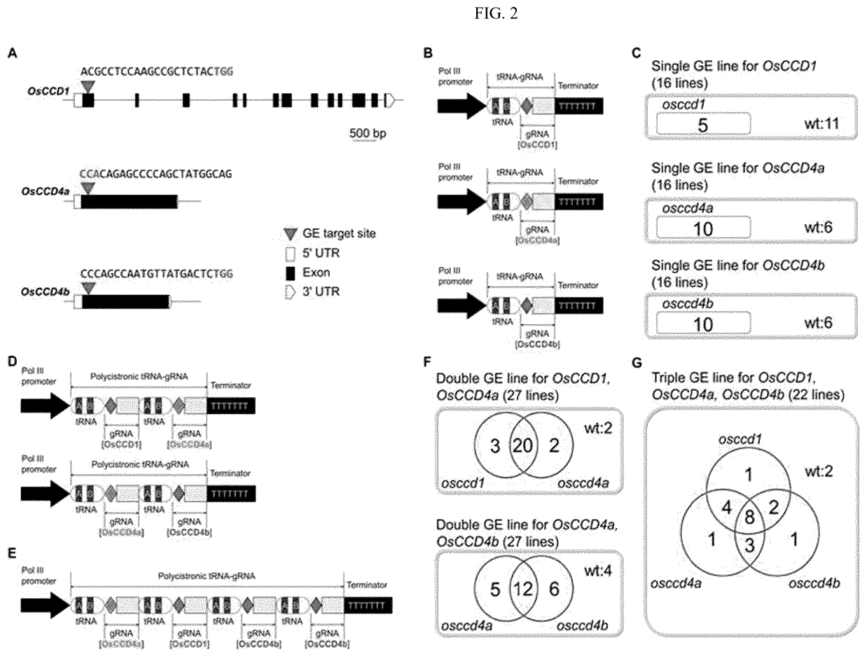

FIG. 2 is a diagram showing single, double, and triple genome editing (GE) line structures for OsCCD1, OsCCD4a, and OsCCD4b:
A: Genomic structures of OsCCD1, OsCCD4a, and OsCCD4b (SEQ ID NOs: 10-12)
B and C: Schematic models of single genome editing (GE) structures
D: Schematic models of double GE structures
E: Schematic models of triple GE structures
F and G: Venn diagrams showing genotypes of double and triple T₀ generations;

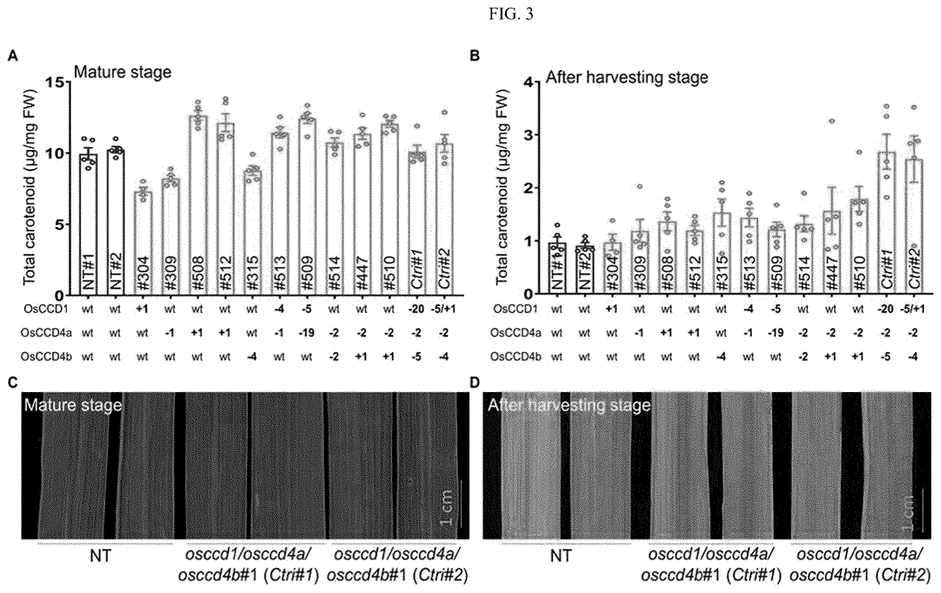

FIG. 3 is a diagram showing total carotenoid contents and colors of various genome-edited KO line rice leaves for OsCCD1, OsCCD4a, and OsCCD4b at a mature green stage before harvest and an aged stage after harvest:
A and B: Total carotenoid contents of leaves before and after harvest
C and D: Phenotypes of leaves before and after harvest of two triple KO lines Ctri #1 and Ctri #2;

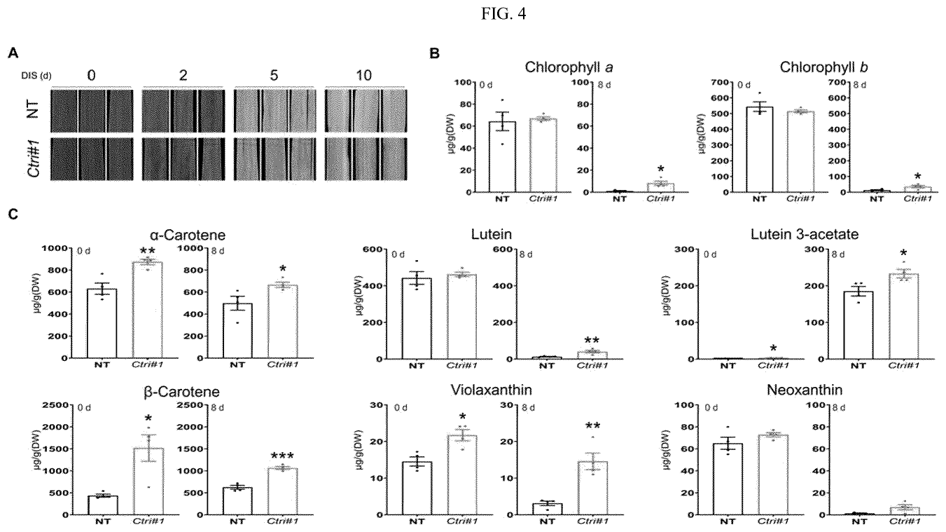

FIG. 4 is a diagram showing changes in color and metabolite content of NT and Ctri #1 by aging treatment induced in a dark environment:
A: Phenotypes for leaf colors
B: Contents of chlorophyll a and chlorophyll b
C: Contents of carotenes (α-carotene and β-carotene), xanthophylls (lutein, violaxanthin, neoxanthin) and carotenoid derivative (lutein 3-acetate);

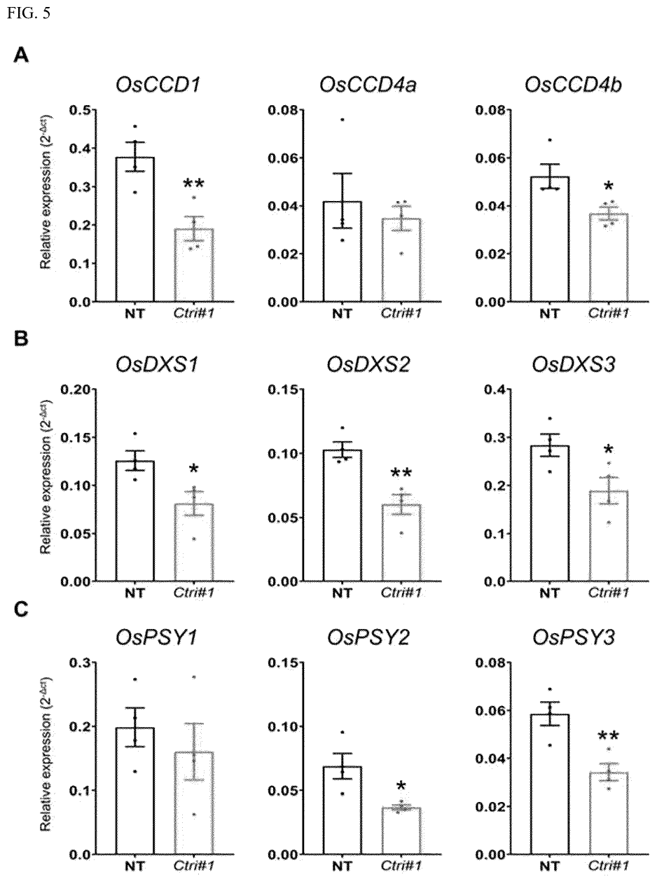

FIG. 5 is a diagram showing transcription levels of OsDXS and OsPSY families, which are major rate-regulating enzyme genes, and three CCDs, which are targets of genome editing, in carotenoid synthesis pathways between NT and Ctri #1 lines;
A: Transcription levels of OsCCD1, OsCCD4a, and OsCCD4b
B: Transcription levels of OsDXS1, OsDXS2, and OsDXS3
C: Transcription levels of OsPSY1, OsPSY2, and OsPSY3;

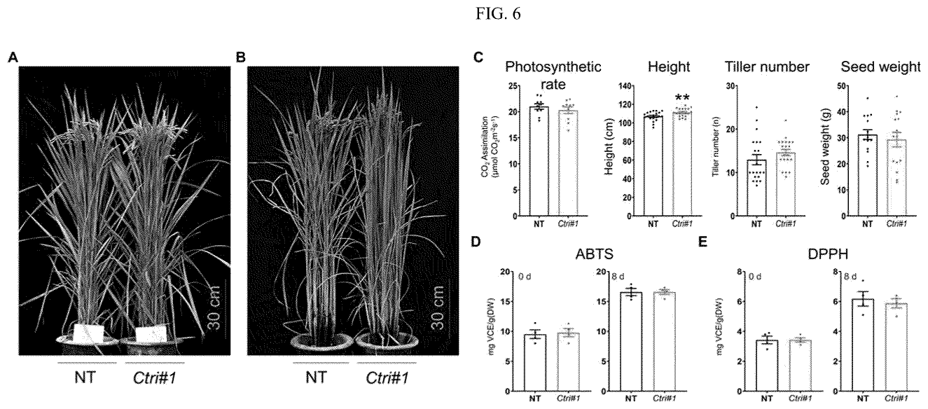

FIG. 6 is a diagram showing agricultural traits and results of ROS scavenging activity analysis in NT and Ctri #1 lines:
A and B: Plant structures at mature green stage (A) and harvesting stage (B) used for agricultural trait evaluation
C: Photosynthetic rate, plant height, tiller number, and harvested seed weight at mature stage using field-cultivated rice plants
D and E: ROS scavenging activity measured using ABTS (D) and DPPH (E) methods before and after aging treatment induced in a dark environment; and

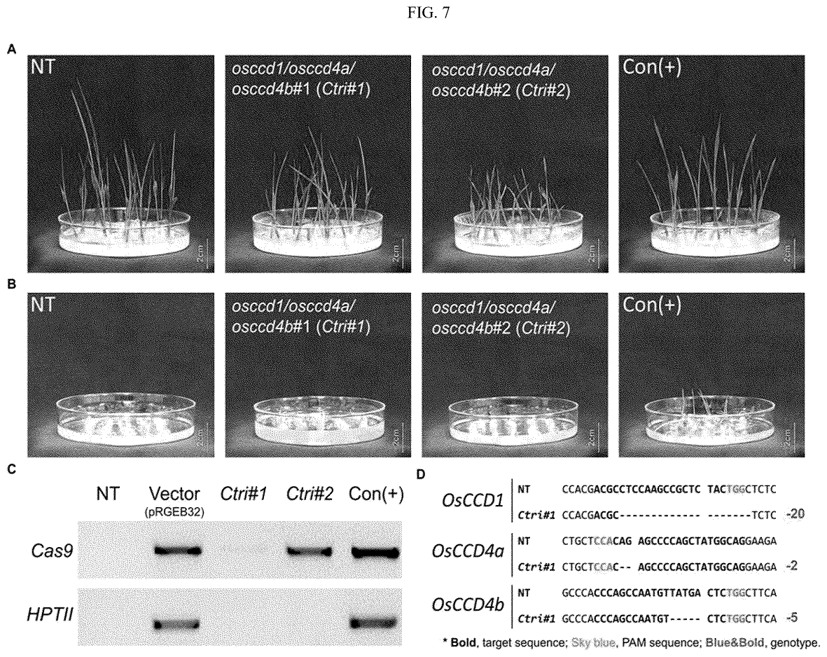

FIG. 7 is a diagram showing selection of T-DNA-free individuals in triple CCD KO (osccd1/osccd4a/osccd4b) rice:
A: Appearances of NT, two osccd1/osccd4a/osccd4b lines (Ctri #1 and Ctri #2), and hygromycin-resistant positive control (Con(+)) cultivated on MSO media, respectively
B: Appearances of NT, two osccd1/osccd4a/osccd4b lines (Ctri #1 and Ctri #2), and hygromycin-resistant positive control (Con(+)) cultivated on hygromycin-containing media
C: PCR results for Cas9 and HPTII genes positioned on LB and RB sides of T-DNA in pRGEB32 vector, respectively
D: Edited genome sequences for gRNA target regions of three CCDs in T-DNA-free Ctri #1 line (SEQ ID NOs: 13-18).

### DETAILED DESCRIPTION OF THE EMBODIMENT

Terminologies used herein are terminologies used to properly express preferred embodiments of the present disclosure, which may vary according to a user, an operator's intention, or customs in the art to which the present disclosure pertains. Accordingly, definitions of the terminologies need to be described based on contents throughout this specification. Throughout this specification, unless explicitly described to the contrary, when a certain part “comprises” a certain component, it will be meant to further comprise other components rather than excluding other components.

Throughout this specification, ‘%’ used to indicate the concentration of a specific material is solid/solid (w/w) %, solid/liquid (w/v) %, and liquid/liquid (v/v) %, unless otherwise stated.

The present disclosure provides transformed rice including a mutation of at least one gene selected from the group consisting of rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes.

In an embodiment of the present disclosure, the OsCCD1 gene may include SEQ ID NO: 1, the OsCCD4a gene may include SEQ ID NO: 2, and the OsCCD4b gene may include SEQ ID NO: 3, but are not limited thereto.

Variants of the base sequences are included within the scope of the present disclosure. A OsCCD1, OsCCD4a or OsCCD4b nucleic acid molecule that may be used as a gene encoding the OsCCD1, OsCCD4a or OsCCD4b protein of the present disclosure is a concept that includes functional equivalents of the nucleic acid molecules that constitute the proteins, for example, variants in which some base sequences of the OsCCD1, OsCCD4a or OsCCD4b nucleic acid molecule have been modified by deletion, substitution or insertion, which have functionally the same function as the OsCCD1, OsCCD4a or OsCCD4b nucleic acid molecule. Specifically, the genes may include base sequences having sequence homology of 70% or more, more preferably 80% or more, much more preferably at 90% or more, and most preferably 95% or more with the base sequence of SEQ ID NO: 1. The “% of sequence homology” with a polynucleotide is confirmed by comparing two optimally aligned sequences with a comparison region, and some of a polynucleotide sequence in the comparison region may include addition or deletion (i.e., gap) compared to a reference sequence (without including addition or deletion) for an optimal alignment of the two sequences.

As used herein, the “transformation” means that DNA is introduced into a host to allow DNA to be replicated as an extrachromosomal factor or by chromosomal integration completion. The transformation includes any method of introducing a nucleic acid molecule into an organism, a cell, a tissue or an organ, and may be performed by selecting a suitable standard technique according to a host cell, as known in the art. Such a method may include electroporation, calcium phosphate (CaPO₄) precipitation, calcium chloride (CaCl₂) precipitation, microinjection, a polyethylene glycol (PEG) method, a DEAE-dextran method, a cationic liposome method, and a lithium acetate-DMSO method, but is not limited thereto.

Since the expression level, modifications, etc. of the protein vary depending on a host cell to be transformed with the expression vector, a host cell most suitable for the purpose may be selected and used. Host cells that may be used in the present disclosure include plant cells, particularly rice (Oryza sativa) cells.

In an embodiment of the present disclosure, the mutation may be a deletion of one or more genes selected from the group consisting of OsCCD1, OsCCD4a and OsCCD4b, preferably a deletion of two or more genes selected from the group consisting of OsCCD1, OsCCD4a and OsCCD4b, and most preferably a deletion of all of the OsCCD1, OsCCD4a and OsCCD4b genes, but is not limited thereto.

In an embodiment of the present disclosure, the transformed rice may be characterized by an enhanced carotenoid content, but is not limited thereto.

In an embodiment of the present disclosure, the carotenoid may be at least one selected from the group consisting of α-carotene, lutein, lutein 3-acetate, β-carotene, violaxanthin, and neoxanthin, but is not limited thereto.

In an embodiment of the present disclosure, the transformed rice may be characterized by a decreased expression of carotenoid biosynthesis genes, but is not limited thereto.

In an embodiment of the present disclosure, the carotenoid biosynthesis gene may be at least one selected from the group consisting of Oryza sativa 1-deoxy-D-xylulose-5-phosphate synthase 1 (OsDXS1), Oryza sativa 1-deoxy-D-xylulose-5-phosphate synthase 2 (OsDXS2), Oryza sativa 1-deoxy-D-xylulose-5-phosphate synthase 3 (OsDXS3), Oryza sativa phytoene synthase 1 (OsPSY1), Oryza sativa phytoene synthase 2 (OsPSY2), and Oryza sativa phytoene synthase 3 (OsPSY3), but is not limited thereto.

In an embodiment of the present disclosure, the deletion of the OsCCD1 gene may be performed using a guide RNA of SEQ ID NO: 4 and a Cas9 protein, the deletion of the OsCCD4a gene may be performed using a guide RNA of SEQ ID NO: 5 and a Cas9 protein, and the deletion of the OsCCD4b gene may be performed using a guide RNA of SEQ ID NO: 6 and a Cas9 protein, but are not limited thereto.

In addition, the present disclosure provides a guide RNA for editing an OsCCD1, OsCCD4a or OsCCD4b gene, which is at least one selected from the group consisting of guide RNAs of SEQ ID NOs: 4 to 9.

In addition, the present disclosure provides a recombinant vector for editing an OsCCD1, OsCCD4a or OsCCD4b gene, including: a sequence encoding the guide RNA; and a sequence encoding the Cas9 protein.

As used herein, the term “vector” refers to a DNA construct including a polynucleotide of a gene operably linked to a suitable regulatory sequence so as to express a target gene in a suitable host. The regulatory sequence may include a promoter capable of initiating transcription, any operator sequence for regulating such transcription, a sequence encoding a suitable mRNA ribosome binding site, and sequences for regulating termination of transcription and translation.

The vector of the present disclosure may include expression regulatory elements such as a promoter, an initiation codon, a termination codon, a polyadenylation signal and an enhancer, a secretion signal, etc., and may be produced in various ways depending on the purpose. The initiation and termination codons need to be functional in a subject when the genetic construct is administered and to be in frame with a coding sequence.

The vector to be used in the present disclosure is not particularly limited as long as the vector is replicable in a host, and may be used with any vector known in the art. For example, non-viral vectors or viral vectors may be used.

A representative example of the non-viral vector is a plasmid. As the plasmid expression vector that may be used in the present disclosure, mammalian expression plasmids known in the art may be used. Representative examples include pRK5 (EP Patent No. 307,247), pSV16B (WO Patent Publication No. 91/08291), pVL1392 (PharMingen), and the like, but are not limited thereto.

In addition, viral vectors may be used as the expression vector of the present disclosure. The viral vectors include, for example, Tobamovirus, Geminivirus, Potexvirus, Caulimovirus, etc. The viral vectors need to meet the following criteria: (1) A viral vector which is able to infect target cells and has an appropriate host range needs to be selected, (2) a transferred gene needs to be able to be preserved and expressed in the cells for an appropriate period of time, and (3) the vector needs to be safe for the host.

A representative example of the Tobamovirus is tobacco mosaic virus (TMV). The TMV may efficiently transfer genes into plant cells and thus is widely used. A representative example of Geminivirus is yellow mosaic virus (YMV). The Geminivirus has double-stranded DNA, which is effective for gene transfer. A representative example of Potyvirus is potato X virus (PVX). The PVX may stably express genes within plant cells. A representative example of Caulimoviruses is cauliflower mosaic virus (CaMV). The CaMV may stably express genes within plant cells.

Other viral vectors that may be used for gene transfer into cells may include other plant viruses, such as tomato yellow leaf curl virus (TYLCV), tobacco ringspot virus (TRSV), tobacco etch mosaic virus (TEV), alfalfa mosaic virus (AMVV), etc.

The expression vector according to the present disclosure may be introduced into plants using methods known in the art. For example, although not limited thereto, the expression vector may be introduced into cells by transient transfection, microinjection, transduction, cell fusion, electroporation, gene gun, and other known methods for introducing nucleic acids into cells (Wu et al., J. Bio. Chem., 267:963-967, 1992; Wu and Wu, J. Bio. Chem., 263:14621-14624, 1988).

In addition, the vector of the present disclosure may further include a selection marker. The selection marker is to confirm whether to select cells transformed with the vector, that is, to insert a target gene, and may be used with markers which give selectable phenotypes, such as drug resistance, auxotrophy, resistance to cytotoxic drugs, or expression of surface proteins. In an environment treated with a selective agent, since only cells expressing the selection marker survive or represent different phenotypes, transformed cells may be screened.

Further, the present disclosure provides a method for producing transformed rice including: editing at least one gene selected from the group consisting of OsCCD1, OsCCD4a, and OsCCD4b by transforming the vector into rice.

In an embodiment of the present disclosure, the transformation may be performed using Agrobacterium, but is not limited thereto.

Hereinafter, the present disclosure will be described in more detail through Examples and Experimental Examples. However, the following Examples and Experimental Examples are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure can be made within the description of claims to be described below and the equivalent scope interpreted therefrom.

### <Preparation Example> Preparation of Plant Materials

Japonica-type Korean rice seeds (Oryza sativa L. cv. Dongjin) were used for transformation. The seeds were sterilized with 70% ethanol and 2% sodium hypochlorite, and then germinated for 1 week using a Murashige-Skoog agar medium in a plant incubator, transplanted to soil, and then cultivated in a paddy field.

### <Example 1> Analysis Method

### 1-1. Binding Energy Analysis Between Carotenoid Ligands and OsCCD

To predict the interaction between each CCD and carotenoid ligands and confirm the binding affinity, the binding energy between carotenoid ligands and OsCCDs (OsCCD1, OsCCD4a, OsCCD4b, OsCCD7, OsCCD8a, and OsCCD8b) was analyzed. The amino acid sequences of the OsCCD proteins were identified through protein data bank (PDB), and 3D structures were constructed using dynamic modeling software. Lycopene, α-carotene, β-carotene, lutein, β-cryptoxanthin, zeaxanthin, violaxanthin, and neoxanthin, which were carotenoid compounds likely to be produced in rice, were selected as candidate carotenoid ligands (substrates), and binding sites of the candidate carotenoid ligands (substrates) and the OsCCDs (OsCCD1, OsCCD4a, OsCCD4b, OsCCD7, OsCCD8a, and OsCCD8b) were predicted and identified using AutoDock software. Thereafter, the binding energy between each carotenoid ligand (substrate) and CCD was calculated.

### 1-2. Genome Editing of OsCCD Gene Group

The CRISPR-Cas9 system was designed by targeting target genes OsCCD1, OsCCD4a, and OsCCD4b.

sgRNAs were designed based on OsCCD gene sequences registered in the NCBI GenBank to target an active site within each gene, and an optimal target region was selected using the web-based tool, CRISPR design tool. An optimal guide including a protospacer associated motif (PAM) sequence was used to guide Cas9 to efficiently bind and cleave.

A single genome editing (GE) line was designed to express the gRNA of each gene under a Pol III promoter (FIG. 2B), a double GE line was designed to express gRNAs of two genes (e.g., OsCCD1 and OsCCD4a) bound to a single vector (FIG. 2D), and a triple GE line was constructed with a vector containing all gRNAs of OsCCD1, OsCCD4a, and OsCCD4b (FIG. 2E). The constructed recombinant vector was transferred to rice cells to generate T₀ generation plants. The genotypes of the generated T₀ generation were analyzed by PCR and sequencing to confirm the success of gene editing.

### 1-3. Method for Analyzing Carotenoids and Chlorophylls in Transformed Rice

For HPLC analysis, extract samples were prepared by adding and then dissolving β-apo-8′-carotenal (0.05 ml, 25 μg ml-1, Sigma-Aldrich) in 50:50 (v/v) dichloromethane/methanol, extracted into layers separated with hexane (1.5 ml) as an internal standard, and dried under liquid nitrogen. Carotenoids were separated on a YMC ODS C-30 column (3 μm, 4.6×250 mm, YMC Europe) using an Agilent 1100 series HPLC system equipped with a photodiode array detector under elution conditions as previously described (Song et al., 2016). For quantitative analysis, HPLC chromatograms generated at 450 nm were used to determine peak areas of calibration curves plotted at four concentrations of β-apo-8′-carotenal and individual carotenoid standards based on peak area ratios. The carotenoid standards were purchased from CaroteNature (Lupsingen, Switzerland) and included α-carotene (β,ε-carotene), 13Z-β-carotene, (all-E)-β-carotene, 9Z-β-carotene, lutein (β,ε-carotene-diol), zeaxanthin (β,β-carotene-diol), β-cryptoxanthin (β,β-caroten-ol), antheraxanthin (dihydro-epoxy-β,β-carotene-diol), and violaxanthin (diepoxy-tetrahydro-β-carotene-diol). The β-carotene content was the sum of the contents of 13Z-β-carotene, (all-E)-β-carotene, and 9Z-β-carotene. Total chlorophyll was extracted from the same T₂ leaf tissues used for siRNA detection and carotenoid analysis, and quantified as chlorophylls a (666 nm) and b (653 nm) on an Optizen POP spectrophotometer (Mecasys Company, Daejeon, Korea) (Song et al., 2016). For statistical analysis, metabolites of all individual samples were quantitatively analyzed three times. Relative differences between groups to non-transformed plants were determined using a two-tailed Student's t-test.

### 1-4. Gene Expression Level Analysis

Transcription levels of an OsCCD gene group and major metabolic enzyme genes were analyzed using qRT-PCR. NT and Ctri #1 (triple OsCCD gene editing line) rice plants were harvested at a mature stage and leaves to be used for analysis were collected. Samples were taken at two time points before and after harvesting. Total RNA was extracted from the collected leaf samples using TRIzol. The quality and concentration of the extracted RNA were confirmed using NanoDrop. Thereafter, a reverse transcription reaction was performed for cDNA synthesis. Primers for OsCCD1, OsCCD4a, OsCCD4b, OsDXS1, OsDXS2, OsDXS3, OsPSY1, OsPSY2, and OsPSY3 were designed based on sequence information registered in Rab-DB (https://rapdb.dna.affrc.go.jp/).

### 1-5. Agricultural Trait Analysis

To perform agricultural trait analysis of genome-edited individuals, plant appearances at mature and harvesting stages were confirmed. In addition, the photosynthetic rate of plants, plant height, tiller number, and weight of harvested seeds were measured.

### 1-6. Analysis of Reactive Oxygen Species (ROS) Scavenging Activity

The reactive oxygen species (ROS) scavenging activity was analyzed using ABTS and DPPH methods. Leaves were collected from plants at the mature and harvesting stages. To obtain total antioxidant extracts, 0.1 g of leaf samples was freeze-pulverized in liquid nitrogen and added with 1 mL of 80% methanol, and the mixture containing methanol was shaken and mixed at room temperature for 30 minutes, and then centrifuged to obtain a supernatant.

### 1) Analysis of ROS Scavenging Activity Using ABTS Method

The ROS scavenging activity of the extract was analyzed using a 2,2′-azinobis(3-ethylbenzothiazoline-6-sulfonic acid) (ABTS) method.

First, an ABTS reaction solution was prepared by mixing ABTS salt and hydrogen peroxide (H₂O₂). A reaction solution was prepared by mixing 2 mM ABTS salt and 2 mM H₂O₂, and then reacted in the dark place for 30 minutes to generate ABTS·+ (ABTS cation free radical).

Thereafter, a plant extract sample was added to the ABTS·+ solution at a certain concentration to neutralize ABTS·+ radicals, and the absorbance was measured at 734 nm to calculate the ROS scavenging activity. The scavenging degree of the ABTS·+ radicals was indicated by a decrease in absorbance. The ROS scavenging activity was calculated using the following Equation.

$$ROS scavenging activity ( \% ) = ( {( absorbance change )}_{control} - {( absorbance change )}_{sample} ) \text{} / {( absorbance change )}_{control} \times 100 \qquad [ Equation 1 ]$$

Through this, the degree to scavenge the ABTS·+ radicals from the plant extract was expressed as a percentage.

### 2) Analysis of ROS Scavenging Activity Using DPPH Method

The ability to scavenge free radicals was analyzed using a diphenylpicrylhydrazyl (DPPH) method.

A DPPH solution was prepared at a concentration of 0.1 mM. The DPPH solution was added with a plant extract at a certain concentration and reacted in a dark place for 30 minutes. Thereafter, the DPPH radical scavenging activity was calculated by measuring the absorbance at 517 nm. A decrease in absorbance values indicated that the DPPH radicals have been scavenged. The scavenging activity of DPPH radicals was calculated using Equation 1 above. This value was expressed as a percentage of the ability to scavenge the DPPH radicals.

### 1-7. Selection of T-DNA-Free Individuals in Triple OsCCD Gene-Editing Lines (Osccd1/Osccd4a/Osccd4b Lines, Ctri #1 and Ctri #2)

Plant seeds of triple OsCCD gene-editing lines Ctri #1 and Ctri #2 induced the growth and development of plants on an MSO medium or an MSO medium containing hygromycin. Thereafter, to confirm the presence of T-DNA, PCR analysis was performed using primers containing left (LB, left border) and right (RB, right border) boundaries of T-DNA in a pRGEB32 vector.

In addition, PCR conditions were set as initial denaturation: 95° C. for 10 minutes/cycle: 95° C. for 30 seconds (denaturation), 58° C. for 30 seconds (binding), 72° C. for 1 minute (elongation)/total 35 repetitions/final elongation: 72° C. for 7 minutes.

Thereafter, PCR analysis for a Cas9 gene was performed to confirm whether the Cas9 protein has been expressed in plant cells. In addition, PCR analysis for a hygromycin-resistant gene (HPTII) was performed to confirm the presence of T-DNA insertion.

### <Example 2> Analysis Results

### 2-1. Binding Energy Analysis Between Carotenoid Ligands and OsCCD

The 3D structure of OsCCD was constructed according to Example 1-1 above, and the binding energy between the candidate carotenoid ligand and OsCCD was analyzed. The analysis results were shown in FIG. 1. Referring to FIG. 1, it was confirmed that the modeled 3D structure consisted of a conical active pocket region and seven β-sheet blades. Among OsCCD1, OsCCD4a, OsCCD4b, OsCCD7, OsCCD8a, and OsCCD8b, OsCCD7, OsCCD8a, and OsCCD8b showed high binding energy with carotenoid ligands, which was confirmed that the carotenoid ligands were located outside the functional pocket of the CCD. Accordingly, genome editing was performed on OsCCD1, OsCCD4a, and OsCCD4b, which showed low binding energy with carotenoid ligands in the OsCCD group, and lycopene, α-carotene, β-carotene, lutein, β-cryptoxanthin, zeaxanthin, violaxanthin, and neoxanthin, which showed low binding energy with OsCCD1, OsCCD4a, and OsCCD4b, were selected as candidate ligands. The sequences of the OsCCD1, OsCCD4a, and OsCCD4b genes were shown in Table 1 below.

TABLE 1

| SEQ ID NO: | Gene | Base sequence (5′→3′) |
|---|---|---|
| 1 | OsCCD1 | ATGGGAGGCGGCGATGGCGATGAGGTGCTGCTGCTGCCGGAGCCG |
| | | CGCCCTCGCAGGGGCCTCGCCTCCTGGGCGCTCGATCTGCTGGAG |
| | | CGCGCCGCCGTCCGCCTCGGCCACGACGCCTCCAAGCCGCTCTAC |
| | | TGGCTCTCCGGCAACTTCGCCCCCGTCCACCACGAGACCCCGCCG |
| | | GCCCCGGCCCTCCCCGTCCGCGGCCACCTCCCCGAGTGCTTGAAT |
| | | GGAGAATTTGTCAGGGTAGGACCCAATCCAAAGTTTGTCCCTGTC |
| | | GCTGGCTATCATTGGTTTGATGGAGATGGAATGATCCATGCGATG |
| | | CGTATCAAAGATGGGAAAGCTACCTATGTGTCGAGATATGTGAAG |
| | | ACTTCTCGTCTCAAGCAAGAAGAGTATTTTGGTGGAGCAAAGTTT |
| | | ATGAAGATTGGAGACCTGAAGGGATTTTATGGATTGTTTATGGTC |
| | | CAAATGCAACAACTCCGGAAAAAACTCAAAGTATTGGATTTTACA |
| | | TATGGACATGGGACAGCTAATACTGCACTTATCTATCACCATGGT |
| | | AAACTTATGGCCCTGTCAGAGGCAGATAAGCCCTATGTTGTTAAG |
| | | GTCCTTGAAGATGGAGACTTGCAAACTCTTGGATTGTTGGATTAC |
| | | GACAAACGGTTGAAACACTCTTTCACTGCTCATCCAAAGGTCGAT |
| | | CCATTTACAGACGAAATGTTCGCTTTTGGATATTCGCATGAACCT |
| | | CCTTACTGTACATACCGGGTCATTACCAAGGATGGAGCCATGCTT |
| | | GATCCTGTGCCAATAACAATTCCAGAATCTGTAATGATGCACGAC |
| | | TTTGCCATTACAGAGAATTATTCTATTTTCATGGACCTTCCTCTG |
| | | TTGTTCCGACCAAAGGAAATGGTGAAGAATGGTGAGTTTATCTAC |
| | | AAGTTTGATCCTACAAAGAAAGCTCGTTTTGGTATACTCCAACGT |
| | | TATGAAAAGGATGACACAAACATCAGATGGTTTGAACTTCCCAAC |
| | | TGCTTCATATTCCACAATGCTAATGCTTGGGAAGAGGGTGACGAA |
| | | GTTATCCTAATTACCTGCCGCTTGGAGAATCCTGACTTGGACAAG |
| | | GTGAACGGTTACCAAAGCGACAATCTCGAGAACTTTGGGAATGAG |
| | | CTGTATGAGATGAGATTCAACATGAAAACCGGTGCTGCTTCACAA |
| | | AAGCAACTATCTGTTTCTGCTGTAGATTTTCCTCGAATTAATGAG |
| | | AGCTACACTGGCAGaaagcagcggtatgtgtattgcgctattcta |
| | | aacagcatagcgaaggtagcaggcattataaaatttgatctacat |
| | | gctgaaccggagatcagtggcatgaaacaacttgaagtgggggga |
| | | aatgtgagaggaatatttgacttgggacctggtagattcgggtcc |
| | | gaggcaatttttgtgcctagggaacccggtgtatctggagaagaa |
| | | gatgatggttatttgatATTCTTTGTCCACGACGAGAATACAGGG |
| | | AAATCTGAAGTCAATGTGATTGATGCAAAGACAATGTCTGCTGAT |
| | | CCGGTGGCAGTTGTTGAGCTACCAAGCCGAGTTCCCTACGGATTC |
| | | CATGCTTTCTTTATAAACGAGGAACAACTGGCAAAACAATCAGCG |
| | | TGA |
| 2 | OsCCD4a | ATGCAAAGGATTTGCCCTGCTCACTGCTCGGTCACTCACTCACTC |
| | | ACCATGAAGTCCATGAGGCTTTCCTACATCCCTCCTGCTGCTTCT |
| | | GCTGCTCCACAGAGCCCCAGCTATGGCAGGAAGAAGAACGCCTCC |
| | | GCCGCTCCGCCATCGGCTGCCGCCTCCACCACCGTTCTCACCTCC |
| | | CCGCTGGTGACCACCACCCGCACTCCGAAGCAGACCGAGCAAGAG |
| | | GACGAGCAGTTGGTAGCCAAGACCAAGACTACGAGAACTGTTATT |
| | | GCTACGACGAATGGCAGGGCGGCGCCGAGCCAGTCTCGGCCTCGC |
| | | CGCCGGCCTGCCCCCGCCGCCGCGGCGTCGGCCGCTTCGCTGCCG |
| | | ATGACGTTCTGCAACGCGCTGGAGGAGGTGATCAACACGTTCATC |
| | | GACCCGCCGGCGCTTCGGCCGGCGGTGGACCCGCGGAACGTGCTG |
| | | ACCAGCAACTTCGTGCCCGTGGACGAGCTGCCGCCGACGCCCTGC |
| | | CCCGTCGTGCGCGGCGCCATCCCGCGCTGCCTCGCCGGCGGCGCC |
| | | TACATCCGCAACGGGCCCAACCCGCAGCACCTCCCGCGCGGGCCG |
| | | CACCACCTGTTCGACGGCGACGGCATGCTGCACTCCCTCCTCCTC |
| | | CCGTCGCCCGCGTCGTCCGGCGACGACCCCGTCCTGTGCTCGCGC |
| | | TACGTGCAGACGTACAAGTACCTCGTGGAGcgcgacgccggcgcg |
| | | cccgtcctgcccaacgtcttctccggcttccacggcgtggccggg |
| | | atggcgcgcggcgccgtcgtggcggccagggtcctgaccgggcag |
| | | atgaatccgttggagggcgtcgggctcgccaacaccagcctcgcc |
| | | tacttcgccggccgcctctacgcgctcggcgagtccgacctcccc |
| | | tacgccgtgcgcgtccacccgGACACCGGCGAGGTGACCACGCAC |
| | | GGCAGGTGCGACTTCGGCGGCCGCCTCGTCATGGGCATGACCGCG |
| | | CACCCCAAGAAGGACCCCGTCACCGGCGAGCTCTTCGCGTTCCGC |
| | | TACGGCCCCGTGCCGCCGTTCGTGACGTACTTCCGGTTCGACCCG |
| | | GCCGGCAACAAGGGCGCCGACGTGCCCATCTTCTCCGTGCAGCAG |
| | | CCGTCGTTCCTGCACGACTTCGCCATCACCGAGCGGTACGCCATC |
| | | TTCCCGGAGATCCAGATCGTGATGAAGCCCATGGACATGGTGGTG |
| | | GGCGGCGGCTCGCCCGTGGGGTCGGACCCCGGCAAGGTGCCCCGC |
| | | CTCGGCGTGATCCCGCGCTACGCCACCGACGAGTCGGAGATGCGG |
| | | TGGTTCGAGGTGCCGGGCTTCAACATCATGCACTCGGTGAACGCG |
| | | TGGGAGGAGGCCGGCGGCGAGGAGCTGGTGCTGGTGGCGCCCAAC |
| | | GTCCTCTCCATCGAGCACGCCCTGGAGCACATGGAGCTAGTGCAC |
| | | TCCTGCGTCGAGAAGGTGCGCATCAACCTCCGCACCGGCGTCGTC |
| | | ACGCGCACCCCGCTCGCCGCCGGGAACTTCGACTTCCCCGTGATC |
| | | AACCCGGCTTTCCTCGGCCGCCGCAACAGGTACGGCTACTTCGGC |
| | | GTCGGCGACCCCGCGCCCAAGATCGGCGGCGTGGCCAAGCTCGAC |
| | | TTCGACCGCGCCGGCGAGGGCGACTGCACCGTGGCGCAGCGCGAC |
| | | TTCGGGCCCGGGTGCTTCGCCGGCGAACCGTTCTTCGTGGCCGAC |
| | | GACGTCGAGGGCAACGGCAACGAGGATGACGGGTACTTGGTGTGC |
| | | TACGTCCACGACGAGGCCACCGGCGAGAACCGGTTCGTGGTGATG |
| | | GACGCGCGGTCGCCGGACCTGGAGATCGTCGCGGAGGTGCAGCTG |
| | | CCCGGACGCGTCCCCTACGGCTTCCATGGCCTGTTCGTCACGCAG |
| | | GCCGAGCTCCAGTCACAGCACCAATGA |
| 3 | OsCCD4b | ATGGAGGTACCCATTGCTGCCATGACTTTTGCCCACCCAGCCAAT |
| | | GTTATGACTCTGGCTTCAAGGCAGCCAAAGAGTAAAAGGTCCCAT |
| | | ATCTCCCCTGCTACCACGGCTCACCGTAATCTACAGACTCGCCTG |
| | | GCTCACCACCACCATGCAACACCAGCTTCATTGCCTATGGCAATC |
| | | TGCAACACAGTAGACAAAGTGATCAATAGGTTCATTGACCTGCCG |
| | | GAGCAGCGACCAACGGTGGATCCGCGGCGTGTGCTCTCTGGCAAC |
| | | TTCGCTCCTGTTGATGAGCTGCCCCCGACAAGCTGCCATGTCATC |
| | | CGCGGCTCCATCCCAAGCTGCCTCGCCGGTGGGGTCTACATCCGC |
| | | AATGGTCCCAACCCACAGCACCGGCTTCCCCAGCGAACACACCAC |
| | | CTCTTCGATGGTGATGGCATGCTCCACTCCCTTCTCATTCCCTCG |
| | | GCCTCGTCAACACTGTTGTCGGAGCCTGTGCTTTGTTCACGCTAT |
| | | GTGCACACGTACAAGTATCTCTTGGAGCGTGAGACCGGAGGACCG |
| | | GTTTTACCAAACTTCTTCGCTGGCTTCCATGGAGTGGCCGGCTTG |
| | | GCTCGTGCAGTGGTCATGATCGCAAGAGTGCTTGCTGGTCAAATT |
| | | AACCTGAACAAGGGCTTCGGGCTGGCCAACACTAGCATCACTCTT |
| | | TTTGCAGATTGCCTATATGCGCTATGCGAATCTGACCTTCCCTAC |
| | | TCCATGCACATCAACCCAGCCAACGGAGAAGTCACCACACTTGGT |
| | | CGATGTGACTTTGGTGGTGATCTTTCTTTTAGGATGACAGCACAC |
| | | CCCAAGAAGGACCCGGTCACCATGGAGTTGTTTGCTTTTCGCTAC |
| | | AATGTCTTCCAACCATTCATAACATACTTCTGGTTCGATCGAGCA |
| | | GGCAGCAAGGTCGCAGATGTGCCCATCTTGTCCTTGCAGAAACCA |
| | | TCGGTGATGCATGACTTTGCAATAACAGAGAGATATGCAATCTTT |
| | | CCAGAGTCACAACTCATCGTTAATCCCATGGACATGGTCATGCGG |
| | | GGGAGCTCGTTGGTAGGATTGGACCGTACCATGGTGCCACGGATT |
| | | GGCGTGCTTCCAAGGTACGCCAAGGATGAGTCAGACATGAGATGG |
| | | TTTGAGGTGCCTAGATTTAATATGTTGCACACGACGAATGGTTGG |
| | | GAAGAGGCTGATGGAGAGGAGATTGTGCTCGTGGCACCCAATATC |
| | | CTATCTATCGAACACATGCTAGGAAACATGGAGCTCATGCGAGCT |
| | | CGTGTCGACATGGTACGTATCAACCTCTGCACCGGTGACGTGTCG |
| | | TGCACTGCACTCTCACCGGAGAGCCTTGAGTTCGGTGTCATCCAC |
| | | CAAGGTTATGTTGGTCGCAAAAATCGCTATGGCTACTTTGGTGTA |
| | | AGTGGTCCGTTGCCCAAGATCAAGGGGATAAGAAAGCTTGACTTT |
| | | GATCTCGTCGGCTCTGGTGATTGCACGGTTGGACGTCGTGACTTT |
| | | GGTCTAGGGTGCTTTGCTGGGGAACCATTTTTTGTTCCAGACAAC |
| | | ATCGACGGGTATGGAAACGAGGATAGTGGTTATGTGGTGTGCTAC |
| | | ACCCATGAAGAGGACACCGGAGAGAGTTGGTTTGTGGTGATGGAT |
| | | GCAAAGTCTCCAGAGCTAGACATTGTTGCAGAAGTGCAACTTCCT |
| | | AGTCGTATCCCCTATGGCTTTCATGGTATTTTTGTCAAACAGGCC |
| | | GAACTTCTCGCACAACAATAA |

### 2-2. Genome Editing of OsCCD Gene Group

The CRISPR-Cas9 system was designed by targeting target genes OsCCD1, OsCCD4a, and OsCCD4b according to Example 1-2 above. FIG. 2 shows sgRNA sequences, genome editing structures, and genotype analysis results. A guide RNA (gRNA) sequence of each gene was designed based on a complementary sequence of a target site and included a PAM sequence (NGG) (FIG. 2A). A single GE line was designed to express the gRNA of each gene under a Pol III promoter (FIG. 2B), a double GE line was designed to express gRNAs of two genes (e.g., OsCCD1 and OsCCD4a) bound to a single vector (FIG. 2D), and a triple GE line was constructed with a vector containing all gRNAs of OsCCD1, OsCCD4a, and OsCCD4b (FIG. 2E). Thereafter, the constructed GE vector was transferred to rice cells to generate T₀ generation plants, and the genotypes of the generated T₀ generation were analyzed by PCR and sequencing to confirm the success of GE. Five in single GE lines for OsCCD1 editing were gene-edited, and ten of single GE lines for OsCCD4a and OsCCD4b editing were gene-edited. In addition, three of double GE lines for OsCCD1 and OsCCD4a editing were gene-edited, and five of double GE lines for OsCCD4a and OsCCD4b editing were gene-edited (FIG. 2F). In addition, eight of triple GE lines of all of OsCCD1, OsCCD4a, and OsCCD4b editing were gene-edited (FIG. 2G). The guide RNA sequences were shown in Table 2 below.

TABLE 2

| SEQ ID NO: | Target gene | Base sequence (5′→3′) |
|---|---|---|
| 4 | OsCCD1 | CAACAAAGCACCAGTGGTCTAGTGGTAGAATAGTACCCTGC |
| | | CACGGTACAGACCCGGGTTCGATTCCCGGCTGGTGCAACGC |
| | | CTCCAAGCCGCTCTACGTTTTAGAGCTAGAAATAGCAAGTT |
| | | AAAATAAGGCTAGTCCGTTATCAACTTGAAAAAGTGGCACC |
| | | GAGTCGGTG |
| 5 | OsCCD4a | CAACAAAGCACCAGTGGTCTAGTGGTAGAATAGTACCCTGC |
| | | CACGGTACAGACCCGGGTTCGATTCCCGGCTGGTGCACTGC |
| | | CATAGCTGGGGCTCTGGTTTTAGAGCTAGAAATAGCAAGTT |
| | | AAAATAAGGCTAGTCCGTTATCAACTTGAAAAAGTGGCACC |
| | | GAGTCGGTG |
| 6 | OsCCD4b | CAACAAAGCACCAGTGGTCTAGTGGTAGAATAGTACCCTGC |
| | | CACGGTACAGACCCGGGTTCGATTCCCGGCTGGTGCACCCA |
| | | GCCAATGTTATGACTCGTTTTAGAGCTAGAAATAGCAAGTT |
| | | AAAATAAGGCTAGTCCGTTATCAACTTGAAAAAGTGGCACC |
| | | GAGTCGGTG |
| 7 | OsCCD1/4a | CAACAAAGCACCAGTGGTCTAGTGGTAGAATAGTACCCTGC |
| | | CACGGTACAGACCCGGGTTCGATTCCCGGCTGGTGCACTGC |
| | | CATAGCTGGGGCTCTGGTTTTAGAGCTAGAAATAGCAAGTT |
| | | AAAATAAGGCTAGTCCGTTATCAACTTGAAAAAGTGGCACC |
| | | GAGTCGGTGCAACAAAGCACCAGTGGTCTAGTGGTAGAATA |
| | | GTACCCTGCCACGGTACAGACCCGGGTTCGATTCCCGGCTG |
| | | GTGCAACGCCTCCAAGCCGCTCTACGTTTTAGAGCTAGAAA |
| | | TAGCAAGTTAAAATAAGGCTAGTCCGTTATCAACTTGAAAA |
| | | AGTGGCACCGAGTCGGTG |
| 8 | OsCCD4a/4b | CAACAAAGCACCAGTGGTCTAGTGGTAGAATAGTACCCTGC |
| | | CACGGTACAGACCCGGGTTCGATTCCCGGCTGGTGCACTGC |
| | | CATAGCTGGGGCTCTGGTTTTAGAGCTAGAAATAGCAAGTT |
| | | AAAATAAGGCTAGTCCGTTATCAACTTGAAAAAGTGGCACC |
| | | GAGTCGGTGCAACAAAGCACCAGTGGTCTAGTGGTAGAATA |
| | | GTACCCTGCCACGGTACAGACCCGGGTTCGATTCCCGGCTG |
| | | GTGCACCCAGCCAATGTTATGACTCGTTTTAGAGCTAGAAA |
| | | TAGCAAGTTAAAATAAGGCTAGTCCGTTATCAACTTGAAAA |
| | | AGTGGCACCGAGTCGGTG |
| 9 | OsCCD1/4a/4b | AAAATAAGGCTAGTCCGTTATCAACTTGAAAAAGTGGCACC |
| | | CAACAAAGCACCAGTGGTCTAGTGGTAGAATAGTACCCTGC |
| | | CACGGTACAGACCCGGGTTCGATTCCCGGCTGGTGCACTGC |
| | | CATAGCTGGGGCTCTGGTTTTAGAGCTAGAAATAGCAAGTT |
| | | GAGTCGGTGCAACAAAGCACCAGTGGTCTAGTGGTAGAATA |
| | | GTACCCTGCCACGGTACAGACCCGGGTTCGATTCCCGGCTG |
| | | GTGCAACGCCTCCAAGCCGCTCTACGTTTTAGAGCTAGAAA |
| | | TAGCAAGTTAAAATAAGGCTAGTCCGTTATCAACTTGAAAA |
| | | AGTGGCACCGAGTCGGTGCAACAAAGCACCAGTGGTCTAGT |
| | | GGTAGAATAGTACCCTGCCACGGTACAGACCCGGGTTCGAT |
| | | TCCCGGCTGGTGCACCCAGCCAATGTTATGACTCGTTTTAG |
| | | AGCTAGAAATAGCAAGTTAAAATAAGGCTAGTCCGTTATCA |
| | | ACTTGAAAAAGTGGCACCGAGTCGGTGCAACAAAGCACCAG |
| | | TGGTCTAGTGGTAGAATAGTACCCTGCCACGGTACAGACCC |
| | | GGGTTCGATTCCCGGCTGGTGCACCCAGCCAATGTTATGAC |
| | | TCGTTTTAGAGCTAGAAATAGCAAGTTAAAATAAGGCTAGT |
| | | CCGTTATCAACTTGAAAAAGTGGCACCGAGTCGGTG |

### 2-3. Analysis Result of Total Carotenoid Content in Genome-Edited Rice

According to Examples 1-3 above, the total carotenoid contents and colors of leaves before and after harvesting were confirmed for five single-edited KO lines (osccd1, osccd4a, osccd4b), five double-edited KO lines (osccd1/osccd4a, osccd4a/osccd4b), and two triple-edited KO lines (osccd1/osccd4a/osccd4b) that were genome-edited KO lines for the OsCCD1, OsCCD4a, or OsCCD4b gene. FIG. 3 showed the results of total carotenoid contents and color analysis results of leaves.

Referring to FIG. 3, in the case of total carotenoid content, in the mature stage, the single KO line showed an increase in carotenoid content of 19 μg/mg FW when OsCCD4a was deleted, so that OsCCD4a deletion had the greatest effect. In addition, the triple KO lines Ctri #1 and Ctri #2 showed an increase of about 20 μg/mg FW compared to a wild type. After the harvesting stage, the triple KO lines Ctri #1 and Ctri #2 showed the most significant effect, with an increase of 2.5 μg/mg FW carotenoid content, respectively.

In terms of leaf colors, the triple KO lines Ctri #1 and Ctri #2 showed dark green leaf colors compared to a wild type (NT) at the mature stage, and the leaves of the triple KO lines Ctri #1 and Ctri #2 appeared yellow after the harvesting stage.

### 2-4. Carotenoid and Chlorophyll Analysis Results of Triple-Edited Rice

The carotenoid and chlorophyll contents of the triple-edited KO line Ctri #1 were confirmed according to Example 1-3 above. NT (wild type) and Ctri #1 lines were treated to induce aging under dark conditions, and changes in the plants were observed at 0, 2, 5, and 10 days after aging treatment. FIG. 4 showed results of the carotenoid content analysis of the triple-edited KO line Ctri #1. Referring to FIG. 4, the triple-edited KO line Ctri #1 showed faster and stronger yellowing than the control group. In addition, the contents of α-carotene, lutein 3-acetate, β-carotene, and violaxanthin in the Ctri #1 line were significantly higher than those in the control group. Therefore, it was shown that the carotenoid content increased during the post-harvest aging process when used as plant feed in farms.

### 2-5. Analysis of Agricultural Traits and ROS Scavenging Activity of Triple-Edited Rice

Agricultural traits and ROS scavenging activity were analyzed in the triple-edited KO line Ctri #1 according to Examples 1-5 and 1-6 above. The analysis results were shown in FIG. 6. Referring to FIG. 6, it was found that the height of rice in the Ctri #1 line significantly increased compared to the control group, but there was no significant difference in other agricultural traits.

### 2-6. Selection of T-DNA-Free Individuals in Triple OsCCD Gene-Edited Lines (Osccd1/Osccd4a/Osccd4b Lines, Ctri #1 and Ctri #2)

According to Example 1-7 above, the Ctri #1 and Ctri #2 lines were cultivated in a general MSO medium and an MSO medium containing hygromycin to confirm the growth states, respectively, and genomic DNA was extracted from each sample to perform PCR targeting the Cas9 gene and HPTII gene included in the pRGEB32 vector to confirm the presence of T-DNA insertion. The presence of T-DNA-free was confirmed as amplification results of the Cas9 and HPTII genes, and T-DNA-free individuals were selected. The analysis results were shown in FIG. 7. Referring to FIG. 7, all samples NT, Ctri #1, Ctri #2, and Con(+) grew normally in the general MSO medium. However, in the MSO medium containing hygromycin, NT, Ctri #1, and Ctri #2 did not grow, whereas Con(+) grew normally (FIGS. 7A and 7B). In addition, the Cas9 and HPTII genes were not detected in Ctri #1 and Ctri #2, whereas the Cas9 and HPTII genes were amplified in Con(+) (FIG. 7C). Through this, it was confirmed that T-DNA was removed from Ctri #1 and Ctri #2.

In addition, as a result of PCR analysis, deletion and substitution were confirmed for each gRNA target sequence in OsCCD1, OsCCD4a, and OsCCD4b gene regions of Ctri #1. Specifically, a 20-bp deletion was found around a target sequence of OsCCD1, a 2-bp deletion was found around a target sequence of OsCCD4a, and a 5-bp deletion was found around a target sequence of OsCCD4b. Through the results, it was confirmed that the Ctri #1 and Ctri #2 lines were T-DNA-free transformed rice in which gene editing was successfully performed.

When describing all of Examples above, it is possible to produce transformed rice in which the OsCCD1, OsCCD4a or OsCCD4b gene is deleted using the CRISPR/Cas9 system, and it is possible to confirm an enhanced carotenoid content in the transformed rice. Therefore, according to the present disclosure, it is possible to produce transformed plants with an enhanced carotenoid content.

What is claimed is:

1. A transformed rice comprising a mutation of at least one gene selected from the group consisting of rice-derived Oryza sativa carotenoid cleavage dioxygenase 1 (OsCCD1), Oryza sativa carotenoid cleavage dioxygenase 4a (OsCCD4a), and Oryza sativa carotenoid cleavage dioxygenase 4b (OsCCD4b) genes.

2. The transformed rice of claim 1, wherein the OsCCD1 gene includes SEQ ID NO: 1, the OsCCD4a gene includes SEQ ID NO: 2, and the OsCCD4b gene includes SEQ ID NO: 3.

3. The transformed rice of claim 1, wherein the mutation is a deletion of at least one gene selected from the group consisting of the OsCCD1, OsCCD4a and OsCCD4b.

4. The transformed rice of claim 1, wherein the transformed rice has an enhanced carotenoid content.

5. The transformed rice of claim 4, wherein the carotenoid is at least one selected from the group consisting of α-carotene, lutein, lutein 3-acetate, β-carotene, violaxanthin, and neoxanthin.

6. The transformed rice of claim 2, wherein the deletion of the OsCCD1 gene is made using a guide RNA of SEQ ID NO: 4 and a Cas9 protein, the deletion of the OsCCD4a gene is made using a guide RNA of SEQ ID NO: 5 and the Cas9 protein, and the deletion of the OsCCD4b gene is made using a guide RNA of SEQ ID NO: 6 and the Cas9 protein.

7. A guide RNA for editing an OsCCD1, OsCCD4a or OsCCD4b gene, which is at least one selected from the group consisting of guide RNAs of SEQ ID NOs: 4 to 9.

8. A recombinant vector for editing an OsCCD1, OsCCD4a or OsCCD4b gene, comprising:
a sequence encoding the guide RNA of claim 7; and
a sequence encoding a Cas9 protein.

9. A method for producing transformed rice comprising:
editing at least one gene selected from the group consisting of OsCCD1, OsCCD4a, and OsCCD4b by transforming the vector of claim 8 into rice.

10. The method of claim 9, wherein the transformation is performed by Agrobacterium.


SEQUENCE LISTING (18 sequences)

SEQ ID NO: 1 - DNA, 1623 bp, synthetic construct
  source 1..1623: mol_type = other DNA
atgggaggcggcgatggcgatgaggtgctgctgctgccggagccgcgccctcgcaggggcctcgcctcctgggcgctcgatctgctggagcgcgccgccgtccgcctcggccacgacgcctccaagccgctctactggctctccggcaacttcgcccccgtccaccacgagaccccgccggccccggccctccccgtccgcggccacctccccgagtgcttgaatggagaatttgtcagggtaggacccaatccaaagtttgtccctgtcgctggctatcattggtttgatggagatggaatgatccatgcgatgcgtatcaaagatgggaaagctacctatgtgtcgagatatgtgaagacttctcgtctcaagcaagaagagtattttggtggagcaaagtttatgaagattggagacctgaagggattttatggattgtttatggtccaaatgcaacaactccggaaaaaactcaaagtattggattttacatatggacatgggacagctaatactgcacttatctatcaccatggtaaacttatggccctgtcagaggcagataagccctatgttgttaaggtccttgaagatggagacttgcaaactcttggattgttggattacgacaaacggttgaaacactctttcactgctcatccaaaggtcgatccatttacagacgaaatgttcgcttttggatattcgcatgaacctccttactgtacataccgggtcattaccaaggatggagccatgcttgatcctgtgccaataacaattccagaatctgtaatgatgcacgactttgccattacagagaattattctattttcatggaccttcctctgttgttccgaccaaaggaaatggtgaagaatggtgagtttatctacaagtttgatcctacaaagaaagctcgttttggtatactccaacgttatgaaaaggatgacacaaacatcagatggtttgaacttcccaactgcttcatattccacaatgctaatgcttgggaagagggtgacgaagttatcctaattacctgccgcttggagaatcctgacttggacaaggtgaacggttaccaaagcgacaatctcgagaactttgggaatgagctgtatgagatgagattcaacatgaaaaccggtgctgcttcacaaaagcaactatctgtttctgctgtagattttcctcgaattaatgagagctacactggcagaaagcagcggtatgtgtattgcgctattctaaacagcatagcgaaggtagcaggcattataaaatttgatctacatgctgaaccggagatcagtggcatgaaacaacttgaagtggggggaaatgtgagaggaatatttgacttgggacctggtagattcgggtccgaggcaatttttgtgcctagggaacccggtgtatctggagaagaagatgatggttatttgatattctttgtccacgacgagaatacagggaaatctgaagtcaatgtgattgatgcaaagacaatgtctgctgatccggtggcagttgttgagctaccaagccgagttccctacggattccatgctttctttataaacgaggaacaactggcaaaacaatcagcgtga

SEQ ID NO: 2 - DNA, 1917 bp, synthetic construct
  source 1..1917: mol_type = other DNA
atgcaaaggatttgccctgctcactgctcggtcactcactcactcaccatgaagtccatgaggctttcctacatccctcctgctgcttctgctgctccacagagccccagctatggcaggaagaagaacgcctccgccgctccgccatcggctgccgcctccaccaccgttctcacctccccgctggtgaccaccacccgcactccgaagcagaccgagcaagaggacgagcagttggtagccaagaccaagactacgagaactgttattgctacgacgaatggcagggcggcgccgagccagtctcggcctcgccgccggcctgcccccgccgccgcggcgtcggccgcttcgctgccgatgacgttctgcaacgcgctggaggaggtgatcaacacgttcatcgacccgccggcgcttcggccggcggtggacccgcggaacgtgctgaccagcaacttcgtgcccgtggacgagctgccgccgacgccctgccccgtcgtgcgcggcgccatcccgcgctgcctcgccggcggcgcctacatccgcaacgggcccaacccgcagcacctcccgcgcgggccgcaccacctgttcgacggcgacggcatgctgcactccctcctcctcccgtcgcccgcgtcgtccggcgacgaccccgtcctgtgctcgcgctacgtgcagacgtacaagtacctcgtggagcgcgacgccggcgcgcccgtcctgcccaacgtcttctccggcttccacggcgtggccgggatggcgcgcggcgccgtcgtggcggccagggtcctgaccgggcagatgaatccgttggagggcgtcgggctcgccaacaccagcctcgcctacttcgccggccgcctctacgcgctcggcgagtccgacctcccctacgccgtgcgcgtccacccggacaccggcgaggtgaccacgcacggcaggtgcgacttcggcggccgcctcgtcatgggcatgaccgcgcaccccaagaaggaccccgtcaccggcgagctcttcgcgttccgctacggccccgtgccgccgttcgtgacgtacttccggttcgacccggccggcaacaagggcgccgacgtgcccatcttctccgtgcagcagccgtcgttcctgcacgacttcgccatcaccgagcggtacgccatcttcccggagatccagatcgtgatgaagcccatggacatggtggtgggcggcggctcgcccgtggggtcggaccccggcaaggtgccccgcctcggcgtgatcccgcgctacgccaccgacgagtcggagatgcggtggttcgaggtgccgggcttcaacatcatgcactcggtgaacgcgtgggaggaggccggcggcgaggagctggtgctggtggcgcccaacgtcctctccatcgagcacgccctggagcacatggagctagtgcactcctgcgtcgagaaggtgcgcatcaacctccgcaccggcgtcgtcacgcgcaccccgctcgccgccgggaacttcgacttccccgtgatcaacccggctttcctcggccgccgcaacaggtacggctacttcggcgtcggcgaccccgcgcccaagatcggcggcgtggccaagctcgacttcgaccgcgccggcgagggcgactgcaccgtggcgcagcgcgacttcgggcccgggtgcttcgccggcgaaccgttcttcgtggccgacgacgtcgagggcaacggcaacgaggatgacgggtacttggtgtgctacgtccacgacgaggccaccggcgagaaccggttcgtggtgatggacgcgcggtcgccggacctggagatcgtcgcggaggtgcagctgcccggacgcgtcccctacggcttccatggcctgttcgtcacgcaggccgagctccagtcacagcaccaatga

SEQ ID NO: 3 - DNA, 1731 bp, synthetic construct
  source 1..1731: mol_type = other DNA
atggaggtacccattgctgccatgacttttgcccacccagccaatgttatgactctggcttcaaggcagccaaagagtaaaaggtcccatatctcccctgctaccacggctcaccgtaatctacagactcgcctggctcaccaccaccatgcaacaccagcttcattgcctatggcaatctgcaacacagtagacaaagtgatcaataggttcattgacctgccggagcagcgaccaacggtggatccgcggcgtgtgctctctggcaacttcgctcctgttgatgagctgcccccgacaagctgccatgtcatccgcggctccatcccaagctgcctcgccggtggggtctacatccgcaatggtcccaacccacagcaccggcttccccagcgaacacaccacctcttcgatggtgatggcatgctccactcccttctcattccctcggcctcgtcaacactgttgtcggagcctgtgctttgttcacgctatgtgcacacgtacaagtatctcttggagcgtgagaccggaggaccggttttaccaaacttcttcgctggcttccatggagtggccggcttggctcgtgcagtggtcatgatcgcaagagtgcttgctggtcaaattaacctgaacaagggcttcgggctggccaacactagcatcactctttttgcagattgcctatatgcgctatgcgaatctgaccttccctactccatgcacatcaacccagccaacggagaagtcaccacacttggtcgatgtgactttggtggtgatctttcttttaggatgacagcacaccccaagaaggacccggtcaccatggagttgtttgcttttcgctacaatgtcttccaaccattcataacatacttctggttcgatcgagcaggcagcaaggtcgcagatgtgcccatcttgtccttgcagaaaccatcggtgatgcatgactttgcaataacagagagatatgcaatctttccagagtcacaactcatcgttaatcccatggacatggtcatgcgggggagctcgttggtaggattggaccgtaccatggtgccacggattggcgtgcttccaaggtacgccaaggatgagtcagacatgagatggtttgaggtgcctagatttaatatgttgcacacgacgaatggttgggaagaggctgatggagaggagattgtgctcgtggcacccaatatcctatctatcgaacacatgctaggaaacatggagctcatgcgagctcgtgtcgacatggtacgtatcaacctctgcaccggtgacgtgtcgtgcactgcactctcaccggagagccttgagttcggtgtcatccaccaaggttatgttggtcgcaaaaatcgctatggctactttggtgtaagtggtccgttgcccaagatcaaggggataagaaagcttgactttgatctcgtcggctctggtgattgcacggttggacgtcgtgactttggtctagggtgctttgctggggaaccattttttgttccagacaacatcgacgggtatggaaacgaggatagtggttatgtggtgtgctacacccatgaagaggacaccggagagagttggtttgtggtgatggatgcaaagtctccagagctagacattgttgcagaagtgcaacttcctagtcgtatcccctatggctttcatggtatttttgtcaaacaggccgaacttctcgcacaacaataa

SEQ ID NO: 4 - RNA, 173 nt, synthetic construct
  source 1..173: mol_type = other RNA
caacaaagcaccagtggtctagtggtagaatagtaccctgccacggtacagacccgggttcgattcccggctggtgcaacgcctccaagccgctctacgttttagagctagaaatagcaagttaaaataaggctagtccgttatcaacttgaaaaagtggcaccgagtcggtg

SEQ ID NO: 5 - RNA, 173 nt, synthetic construct
  source 1..173: mol_type = other RNA
caacaaagcaccagtggtctagtggtagaatagtaccctgccacggtacagacccgggttcgattcccggctggtgcactgccatagctggggctctggttttagagctagaaatagcaagttaaaataaggctagtccgttatcaacttgaaaaagtggcaccgagtcggtg

SEQ ID NO: 6 - RNA, 173 nt, synthetic construct
  source 1..173: mol_type = other RNA
caacaaagcaccagtggtctagtggtagaatagtaccctgccacggtacagacccgggttcgattcccggctggtgcacccagccaatgttatgactcgttttagagctagaaatagcaagttaaaataaggctagtccgttatcaacttgaaaaagtggcaccgagtcggtg

SEQ ID NO: 7 - RNA, 346 nt, synthetic construct
  source 1..346: mol_type = other RNA
caacaaagcaccagtggtctagtggtagaatagtaccctgccacggtacagacccgggttcgattcccggctggtgcactgccatagctggggctctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaacgcctccaagccgctctacgttttagagctagaaatagcaagttaaaataaggctagtccgttatcaacttgaaaaagtggcaccgagtcggtg

SEQ ID NO: 8 - RNA, 346 nt, synthetic construct
  source 1..346: mol_type = other RNA
caacaaagcaccagtggtctagtggtagaatagtaccctgccacggtacagacccgggttcgattcccggctggtgcactgccatagctggggctctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacccagccaatgttatgactcgttttagagctagaaatagcaagttaaaataaggctagtccgttatcaacttgaaaaagtggcaccgagtcggtg

SEQ ID NO: 9 - RNA, 692 nt, synthetic construct
  source 1..692: mol_type = other RNA
caacaaagcaccagtggtctagtggtagaatagtaccctgccacggtacagacccgggttcgattcccggctggtgcactgccatagctggggctctggttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaacgcctccaagccgctctacgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacccagccaatgttatgactcgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcacccagccaatgttatgactcgttttagagctagaaatagcaagttaaaataaggctagtccgttatcaacttgaaaaagtggcaccgagtcggtg

SEQ ID NO: 10 - DNA, 23 bp, synthetic construct
  source 1..23: mol_type = other DNA
acgcctccaagccgctctactgg

SEQ ID NO: 11 - DNA, 23 bp, synthetic construct
  source 1..23: mol_type = other DNA
ccacagagccccagctatggcag

SEQ ID NO: 12 - DNA, 23 bp, synthetic construct
  source 1..23: mol_type = other DNA
cccagccaatgttatgactctgg

SEQ ID NO: 13 - DNA, 33 bp, synthetic construct
  source 1..33: mol_type = other DNA
ccacgacgcctccaagccgctctactggctctc

SEQ ID NO: 14 - DNA, 13 bp, synthetic construct
  source 1..13: mol_type = other DNA
ccacgacgctctc

SEQ ID NO: 15 - DNA, 33 bp, synthetic construct
  source 1..33: mol_type = other DNA
ctgctccacagagccccagctatggcaggaaga

SEQ ID NO: 16 - DNA, 31 bp, synthetic construct
  source 1..31: mol_type = other DNA
ctgctccacagccccagctatggcaggaaga

SEQ ID NO: 17 - DNA, 33 bp, synthetic construct
  source 1..33: mol_type = other DNA
gcccacccagccaatgttatgactctggcttca

SEQ ID NO: 18 - DNA, 28 bp, synthetic construct
  source 1..28: mol_type = other DNA
gcccacccagccaatgtctctggcttca